(19) United States
(12) Patent Application Publication
(10) Pub. No.: US 2021/0356377 A1
(43) Pub. Date: Nov. 18, 2021

(54) Methods Of Cell Selection

(71) Applicant: MedImmune, LLC, Gaithersburg, MD (US)

(72) Inventors: Zachary BRITTON, Gaithersburg, MD (US); Marcello MARELLI, Gaithersburg, MD (US); Michael A. BOWEN, Gaithersburg, MD (US); Timothy LONDON, Cambridge (GB); Li ZHUANG, Gaithersburg, MD (US); Lina CHAKRABARTI, Gaithersburg, MD (US)

(21) Appl. No.: 17/274,042

(22) PCT Filed: Sep. 5, 2019
(86) PCT No.: PCT/US19/49745

§ 371 Date: Mar. 5, 2021

Related U.S. Application Data
(60) Provisional application No. 62/728,186, filed on Sep. 7, 2018.

(51) Int. Cl. G01N 15/14; C12N 5/00; C12N 9/00; C07K 14/705; G01N 33/58

(52) U.S. Cl. CPC G01N 15/14; C12N 5/0018; C12N 9/93; C07K 14/705; C07K 2319/30; C12N 2500/33; C12Y 601/01026; C07K 2319/03; C07K 2319/912; G01N 33/582

(57) ABSTRACT

The present invention is directed to methods of screening populations of transgenic cells for cells that produce a protein of interest. The methods comprise culturing transgenic cells in culture conditions that include at least one non-natural amino acid (nnAA) in the cell culture medium. The transgenic cells comprise at least one polynucleotide that codes for a fusion protein with a first domain coding for a protein of interest and a second domain coding for a domain that facilitates detection of the transgenic cells that express the protein of interest when the transgenic cell expresses the second domain.

### BACKGROUND OF THE INVENTION

### Field of the Invention

The present invention is directed to methods of screening populations of transgenic cells for cells that produce a protein of interest. The methods comprise culturing transgenic cells in culture conditions that include at least one non-natural amino acid (nnAA) in the cell culture medium. The transgenic cells comprise at least one polynucleotide that codes for a fusion protein with a first domain coding for a protein of interest and a second domain coding for a domain that facilitates detection of the transgenic cells that express the protein of interest when the transgenic cell expresses the second domain. The fusion protein also comprises at least one nnAA.

### Background of the Invention

Transgenic cells producing a protein of interest are used throughout drug development. For example, in the early parts of drug development, such cells are used for antibody generation campaigns and cell-based assays to assess activity, and in later parts of drug development, such cells are used to produce a biopharmaceutical. Therefore, transgenic cell selection is critical for drug development.

There have been many efforts to provide alternative methods for selecting transgenic cells that produce a protein of interest. For example, flow cytometry has made it more feasible to identify cells with specific characteristics. Important advantages of flow cytometry include the ability to screen large numbers of cells. However, flow cytometry is time and labor intense.

The present invention provides advantages over existing methods by, for example, reducing workload and increasing efficiency. For example, PCT Publication No. WO 2003/014361 discloses a system using stop codon suppression technology to enable read-through and expression of fusion proteins, with one of the domains of the fusion protein being a gene for antibiotic resistance. The cells are then cultured in the presence of the antibiotics to determine which cells expression the fusion. This method, however, does not allow for selection of high level fusion protein producers, thus there is no mechanism for selecting the high producing clones. Similarly, PCT Publication No. WO 2005/073375 discloses the use of an antibiotic-dependent system such that the transgenic cells much be exposed to antibiotics for at least two days in culture before stop codon suppression can be achieved. These methods also do not result in the ability to select for high producers after a single round of cloning/selection. PCT Publication No. WO 2010/022961 utilizes a codon sequence that enables leaky read-through of stop codons. Such a system is not inducible, exhibits low efficiency and is not capable of being tightly controlled. Thus, this system is also incapable of selecting high producers of the protein of interest after a single round of cloning and selection.

The present invention provides a novel approach for enriching and selecting transgenic cells with many advantages, including increased efficiency and control. This approach has also been validated across very different proteins of interest and as such can be used as a platform to enrich and select transgenic cells expressing any protein of interest.

### SUMMARY OF THE INVENTION

The present invention is directed to methods of screening populations of transgenic cells for cells that produce a protein of interest. The methods comprise culturing transgenic cells in culture conditions that include at least one non-natural amino acid (nnAA) in the cell culture medium. The transgenic cells comprise at least one polynucleotide that codes for a fusion protein with a first domain coding for the protein of interest and a second domain coding for a domain that facilitates detection of the transgenic cells that express the protein of interest when the transgenic cell expresses the second domain. The fusion protein also comprises at least one nnAA.

In one embodiment, the transgenic cells comprise at least one polynucleotide that codes for a fusion protein with a first domain coding for the protein of interest and a second domain coding for an anchor domain that anchors the protein of interest to the cell membrane when the transgenic cell expresses the second domain. These fusion proteins also comprise at least one nnAA.

In another embodiment, the transgenic cells comprise at least one polynucleotide that codes for a fusion protein with a first domain coding for the protein of interest and a second domain coding for a tag that labels the protein of interest when the transgenic cell expresses the second domain. These fusion proteins also comprise at least one nnAA.

### BRIEF DESCRIPTION OF THE DRAWINGS

FIG. 1 depicts the inducible system used in the methods of the present invention. The presence of nnAA results in the readthrough of an amber stop codon encoded at the 3′ end of the target gene and the expression of a fusion protein, for example, an antibody containing a transmembrane domain or glycosylphosphatidyl inositol (GPI) attachment sequence that enables the anchoring of the antibody to a cell membrane. In these same cells, the absence of nnAAs in the cell culture medium results in the expression of the antibody without the membrane anchor and is secreted into the cell culture medium. In these embodiments, the codon coding for the nnAA is at the C-terminus of the heavy chain of the antibody, which permits the anchoring antibody, via the heavy chain, onto the cell membrane.

FIG. 2 depicts polynucleotides that can be inserted into the transgenic cells of the present invention. In this embodiment, HC represents the heavy chain of the antibody and DAF-7 represents a GPI signal peptide that allows anchoring of the heavy chain onto the cell surface membrane. TM represent a transmembrane domain from a transmembrane protein. In this example, the TM domain of human thrombomodulin was used. TAG (red stars) represents amber stop codons that encode a nnAA when it is present in the culture medium. TGA and TAA (green stars) represent stop codons that are not integration sites for the nnAA.

FIG. 3 depicts flow cytometry results using the methods of the present invention. Cells expressing the indicated constructs were grown in the presence, or absence, of nnAA and the surface bound IgG detected with anti-HC and anti-LC antibodies. The construct “IgG-DAF-Amber” shows low surface fluorescence in the absence of nnAAs, but high surface fluorescence in the presence of nnAA in the cell culture medium. The construct “IgG-TM-1×Amber” shows low surface fluorescence in the absence of nnAAs, but high surface fluorescence in the presence of nnAA in the cell culture medium.

FIG. 4 depicts the correlations of MFI and expression titres of expression pools selected after activation of the surface display system with 2 mM, 0.0 mM and 0.1 mM nnAA. The correlation between expression and MFI obtained by surface display was improved with lower concentrations of the nnAA. B) The extent of surface display of the antibody-GPI fusion can be tuned by modulating the concentration of nnAA and the time of exposure of the cells to the nnAA. With longer exposures or increased nnAA concentration the MFI of the population increases.

FIG. 5 depicts the Surface display-based selection of stable pools. A) sorting gates of cells stably expressing an IgG-GPI-Amber were grown in the absence (−nnAA) or presence of nnAA (+nnAA) and sorted into pools by FACS based on HC expression to segregate cells into a non-enriched, low (bottom) and high (top) surface display pools. B) Sorted sub-pools were expanded and their fed-batch titers and C) specific productivity (Qp) were determined. Cells sorted from the high surface display showed higher overall titers than non-enriched or low surface display pools.

FIG. 6 depicts that high surface display levels correlate with increased titers. A) Stable IgG-GPI-Amber pools were grown in the presence of nnAA and bulk sorted by FACS into low, medium and high surface staining pools. B) Expression levels of the sorted sub-pools by fed-batch cultures were determined (n=3). C) surface display of the sorted sub-pools was measured by flow cytometry after expansion. Three overlapping populations with different surface staining levels were preserved through the fed-batch process. D) Heavy chain surface display MFI and sub-pool titers shows a strong positive correlation. Coefficient of determination (R²) is shown.

FIG. 7 depicts the utility of surface display for the identification and isolation of high expression clones. A) Single cells expressing the IgG-GPI-Amber were sorted from high, medium and low surface display gates using surface display and cells sorted into 96 well plates at single cell density. Expression levels of these clones were measured in 96 deep well fed-batch cultures. The expression levels of each clone were plotted along with a control population sorted in the absence of surface display. Mean expression values from high surface display gates showed a statistically significant difference to medium, low and non-enrich populations. P values for the indicated pairs is shown. B) To highlight the enrichment of high producing clones using surface display the frequency of clones above the selected titres were plotted in a stacked graph. High surface display selections showed an enrichment in very high producing clones (>8.5 g/L) than other gates or the non-enrich population. C) The correlation between surface display MFI and expression levels was examined in 30 clones from each of the surface display gated fractions. A fitted regression line is shown with its coefficient of determination (R²). These data show a strong correlation between surface display and expression titer.

FIG. 8 depicts the utility of surface display for the enrichment and selection of high expressors for difficult to express molecules. A) Cells stably expressing a bispecific antibody, which has shown low titres in conventional cell line engineering screens, were subjected to surface display and high and low gates used to select single cells. Clones derived from a non-enriched population were also isolated. B) Isolated clones were assessed for expression titer in 96 deep well fed-batch culture. Clones derived from a high surface display gate show higher overall expression titer levels, but also higher numbers of high producing clones.

FIG. 9 depicts flow cytometry measurement of amber suppression in Jump-In CHOK1 cell lines. Representative data for Pool 1 transfected with a mCherry_{AMB}GFP reporter (9A). Clone 7 transfected with a mCherry_{AMB}GFP reporter (9B).

FIG. 10 depicts DNA and protein constructs for validation of this (inducible) approach. DNA constructs representing amber suppression-dependent (AMB) and read-through (K) variants. AzK, lysine analogue incorporated by pylRS/tRNA pair (10A). Proteins representing amber suppression-dependent (AMB) and read-through (K) variants with and without nnAA supplementation (10B). In the absence of the nnAA, cells containing the AMB construct express only ‘untagged’ protein variants; however, in the presence of the nnAA, cells express both ‘untagged’ and ‘tagged’ protein variants. Cells containing the read-through construct only express the ‘tagged’ protein variant. Internal Rho 1D4 sequences (*) are not recognized by the Rho 1D4 antibody.

FIG. 11 depicts Western blots demonstrating that tagged membrane protein expression is elicited specifically by exposure of cells to nnAA. EphA2-, Claudin 1-, CXCR2- or CXCR4-expressing cells were supplemented with nnAA to induce expression of GFP. Total cellular lysates were generated at 0, 24, and 48 h post-exposure to nnAA and evaluated by Western blotting using antibodies directed against Rho 1D4, eGFP, and tubulin. Arrows indicate ‘untagged’ membrane protein (single star) and ‘tagged’ membrane protein (double star). The ‘read-through’ variant for each membrane protein (not pictured) was used to identify the ‘tagged’ variants.

FIG. 12 depicts the comparative analysis of parental, pre-sorted and sorted populations. Total expression of ‘untagged’ EphA2, Claudin 1, CXCR2, and CXCR4 in parental, pre-sorted and sorted populations was evaluated by Western blot (12A). Cell surface FCM using the anti-EphA2 1C1 (12B). Cell surface FCM using the anti-Claudin 1 FAB4618R (12C). Cell surface FCM using the anti-CXCR2-X2-753 (12D). Cell surface FCM using the anti-CXCR4 MEDI3185 (12E). IL-8 ligand binding to parental and sorted ‘untagged’ CXCR2-expressing cell lines (12F). SDF-1α ligand binding to parental and sorted ‘untagged’ CXCR4-expressing cell lines (12G).

FIG. 13 depicts fluorescence microscopy of membrane protein cell lines at 0 and 48 h post-nnAA exposure. Clone 7 cells expressing EphA2-, Claudin 1-, CXCR2- or CXCR4 were evaluated by fluorescence microscopy 48 h post-exposure to nnAA. The ‘tagged’ variants for each membrane protein exhibited similar cellular distributions as the ‘read-through’ variants. Claudin 1 fusion proteins show localization to areas of cell-to-cell contacts. Arrows indicate cell-to-cell interactions.

### DETAILED DESCRIPTION OF THE INVENTION

The present invention is directed to methods of screening populations of transgenic cells for cells that produce a protein of interest. The methods comprise culturing transgenic cells in culture conditions that include at least one non-natural amino acid (nnAA) in the cell culture medium. The transgenic cells comprise at least one polynucleotide that codes for a fusion protein with a first domain coding for the protein of interest and a second domain coding for a domain that facilitates detection of the transgenic cells that express the protein of interest when the transgenic cell expresses the second domain. The fusion protein also comprises at least one nnAA.

In one specific embodiment, the present invention is directed to methods of screening populations of transgenic cells for cells that produce higher levels of a protein of interest as compared to other cells producing lower levels of the protein of interest within the population of transgenic cells. The methods comprise culturing transgenic cells in culture conditions that include at least one non-natural amino acid (nnAA) in the cell culture medium. The transgenic cells comprise at least one polynucleotide that codes for a fusion protein with a first domain coding for the protein of interest and a second domain coding for an anchor domain that anchors the protein of interest to the cell membrane when the transgenic cell expresses the second domain. These fusion proteins also comprise at least one nnAA.

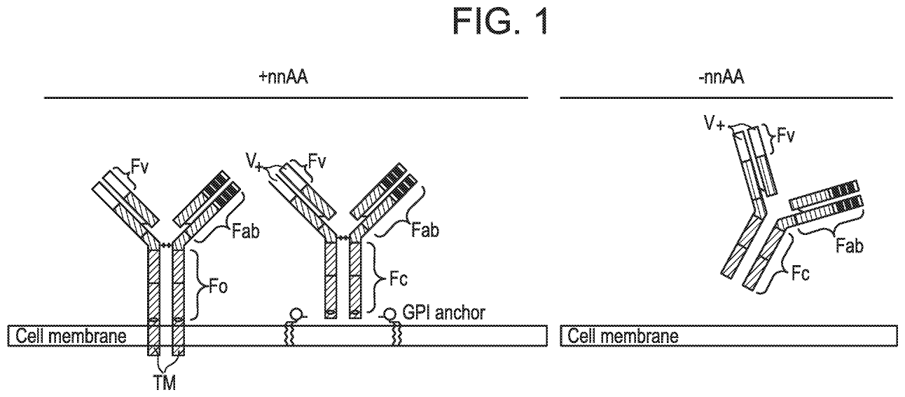

In another specific embodiment of the invention, FIG. 1 depicts an inducible system used in the methods of the present invention. The presence of nnAA results in the readthrough of an amber stop codon encoded at the 3′ end of the target gene and the expression of a fusion protein, for example, an antibody containing a transmembrane domain or glycosylphosphatidyl inositol (GPI) attachment sequence that enables the anchoring of the antibody to a cell membrane. In these same cells, the absence of nnAAs in the cell culture medium results in the expression of the antibody without the membrane anchor and is secreted into the cell culture medium. In these embodiments, the codon coding for the nnAA is at the C-terminus of the heavy chain of the antibody, which permits the anchoring antibody, via the heavy chain, onto the cell membrane.

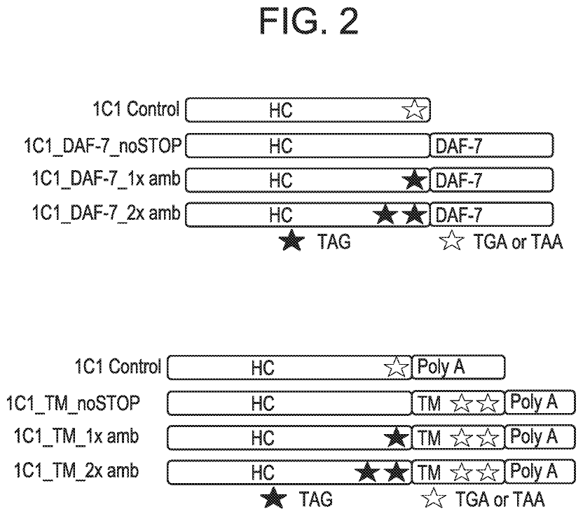

In another specific embodiment of the invention, FIG. 2 depicts polynucleotides that can be inserted into the transgenic cells of the present invention. In this embodiment, HC represents the heavy chain of the antibody and DAF-7 represents a GPI signal peptide that allows anchoring of the heavy chain onto the cell surface membrane. TM represent a transmembrane domain from a transmembrane protein. In this example, the TM domain of human thrombomodulin was used. TAG (red stars) represents amber stop codons that encode a nnAA when it is present in the culture medium. TGA and TAA (green stars) represent stop codons that are not integration sites for the nnAA.

In another specific embodiment, the present invention is directed to methods of screening populations of transgenic cells for cells that produce a protein of interest containing at least one transmembrane domain. The methods comprise culturing transgenic cells in culture conditions that include at least one non-natural amino acid (nnAA) in the cell culture medium. The transgenic cells comprise at least one polynucleotide that codes for a fusion protein with a first domain coding for the protein of interest and a second domain coding for a tag that labels the protein of interest when the transgenic cell expresses the second domain. These fusion proteins also comprise at least one nnAA.

The transgenic cells of the present invention have been engineered to incorporate nnAAs into a polypeptide chain when the nnAAs are present in the cell culture medium. As used herein, the term non-natural amino acid (nnAA) is used to mean any molecule with the structure of an amino acid that is not one of the 20 “proteinogenic” amino acids of the standard genetic code. One of skill in the art fully recognizes the 20 proteinogenic amino acids of the standard genetic code, thus one of skill in the art would readily recognize an amino acid that is a nnAA. Specifically, the term nnAA does not include the following amino acids: arginine, lysine, aspartic acid, glutamic acid, glutamine, asparagine, histidine, serine, threonine, tyrosine, cysteine, tryptophan, alanine, isoleucine, leucine, methionine, phenylalanine, valine, proline, glycine. The term nnAA, however, may include derivatives of the above-listed amino acids, provided that these derivatives are not any of the 20 proteinogenic amino acids of the standard genetic code. Importantly, the specific identity of the nnAA is not critical to the methods of the present invention, provided that the nnAA can be incorporated into a growing polypeptide chain and can support amber suppression. Instead, the mere presence or absence of an nnAA in the transgenic cell culture environment is critical, provided that the nnAA can be incorporated into a growing polypeptide chain. Examples of nnAAs that can be used in the methods of the present invention include but are not limited to those nnAAs listed in Liu, C. and Shultz, P., Ann. Rev. Biochem., 79:413-444 (2010) and Wan et al. Biochim Biophys Acta. 2014 June; 1844(6): 1059-1070, which are incorporated by reference.

In one specific embodiment, the nnAA present in the cell culture environment in the methods of the present invention is a lysine analogue (including, but not limited to, pyrrolysine, lysine azide, propargyl lysine, lysine-Aloc, and Boc-lysine). The identity of the nnAA to be used in the methods of the present invention would necessarily identify the orthogonal tRNA and orthogonal tRNA synthetase. For example, when the nnAA of the present invention is lysine azide, the orthogonal tRNA synthetase would be identified pyrrolysyl-tRNA synthetase or PylRS, and its cognate tRNA as tRNA-Pyl (or tRNA(Pyl)). Thus, in one embodiment, the nnAA is lysine azide and the orthogonal tRNA synthetase is pyrrolysyl-tRNA synthetase and the tRNA is tRNA-Pyl.

In another specific embodiment, certain naturally occurring amino acids may be used in the methods of the present invention. In such cases, the nnAA as described or claimed herein will also include those certain naturally occurring amino acids. Such naturally occurring amino acids, include, but are not limited to, pyrrolysine.

It is now well-known in the art how to construct cells that have the ability to incorporate nnAAs into the amino acid sequences of peptides when these peptides are being produced through normal cellular transcription and translation processes. Namely, cells that have the ability to incorporate nnAAs into growing peptide strands must include orthogonal tRNAs that have been engineered to “accept” the nnAA and a tRNA synthetase that is “matched” to the orthogonal tRNA and the nnAA. The orthogonal tRNA synthetase “attaches” the nnAA to the orthogonal tRNA during the esterification reaction to form an aminoacyl-tRNA with the nnAA. As used herein, the term “orthogonal tRNA synthetase” is used as it is in the art to mean a species of tRNA synthetase that is normally not present in the specific cell type being cultured. Normally, the orthogonal tRNA synthetase has specificity for a nnAA and would not accept any naturally occurring amino acid during the esterification process. In addition, the “orthogonal tRNA” is not recognized by the host cell's tRNA synthetases. But the transgenic cells of the present invention have been engineered to express both the orthogonal tRNA synthetase and tRNA such that the orthogonal tRNAs will be loaded with nnAAs. Any known orthogonal tRNA synthetase and tRNA pairs can be used in the present invention so long as the tRNA can be loaded with the nnAA to incorporate and support amber suppression.

During the operation of the methods of the present invention, the orthogonal tRNA and matched orthogonal tRNA synthetase will insert the matched nnAA into a growing peptide chain at a site designated by an amber stop codon, when the nnAA is present in the culture environment. When the nnAA is absent from the culture environment, the orthogonal tRNA will not accept any naturally occurring amino acid and thus the amber codon will function as a stop codon and result in the halt of peptide synthesis. In other words, the orthogonal tRNAs present in the transgenic cells used in the methods of the present invention are engineered such that the anti-codon loop of the tRNA will base pair to a stop codon on an mRNA molecule. The presence of the nnAA in the culture conditions with the transgenic cells will thus permit elongation of the growing polypeptide chain in the transgenic cell whereby the nnAA is incorporated into the growing polypeptide chain. Moreover, the absence of the nnAA in the culture conditions with the transgenic cells will cause the polypeptide chain to stop growing since no amino acid would be inserted into the polypeptide chain beyond the amber stop codon. The presence or absence of the nnAA in the culture conditions with the transgenic cells thus acts as a “gatekeeper” for polypeptide elongation. Accordingly, the specific identity of the nnAA is not critical for the operation of the methods of the present invention, since the nnAA is merely acting as gatekeeper for polypeptide elongation.

The transgenic cells of the present invention can be any cell type capable of being cultured and capable of being engineered to generate the orthogonal tRNA and the matched orthogonal tRNA synthetase. Examples of cells that can be used in the methods of the present invention include but are not limited to eukaryotic cells such as but not limited to mammalian cells, insect cells and yeast cells and prokaryotic cells such as bacterial cells. Specific examples of transgenic cells that can be used in the methods of the present invention include but are not limited to E. coli cells, CHO cells, HEK293 cells, PERC6 cells, COS-1 cells, HeLa cells, VERO cells and mouse hybridoma cells. In one embodiment, the cells disclosed in PCT Publication No. WO 2014/044872 can be used for the methods of the present invention.

The transgenic cells of the present invention also comprise at least one polynucleotide that codes for a fusion protein at least a first domain coding for a protein of interest and at least a second domain coding for a domain that facilitates detection of the transgenic cells expressing the protein of interest in or on the transgenic cells. The polynucleotide that codes for the fusion protein of the first and second domains also comprises at least one codon that codes for the nnAA. As explained above, when the nnAA is present during the culture conditions, the polypeptide chain will continue to elongate during protein synthesis such that the first and second domain are both generated during protein synthesis, wherein these first and second domains are separated by at least one nnAA. In one specific embodiment, the polynucleotide codes for more than one nnAA. In a more specific embodiment, these multiple nnAAs are the same nnAA. In another more specific embodiment, the multiple nnAAs are different nnAAs. If more than two nnAAs are coded for in the polynucleotide, two or more of the nnAAs may be the same or different from one another. This first codon that codes for the first nnAA in between the first and second domains will also serve as a stop codon during protein synthesis such that the polypeptide chain stops growing after the first domain is generated.

In select embodiments, the polynucleotide coding for the fusion protein also codes for a linker peptide between the first and second domains. In these embodiments, the codon coding for at least one nnAA may be 5′ or 3′ to the linker peptide. As used herein, a linker peptide is a used to mean a polypeptide typically ranging from about 1 to about 120 amino acids in length that is designed to facilitate the functional connection of two domains into a linked binding domain. To be clear, a single amino acid can be considered a linker peptide for the purposes of the present invention. Of course, the linker peptides used in the fusion proteins of the present invention may comprise or in the alternative consist of amino acids numbering more than 120 residues in length. The length of the linker peptide, if present, may not be critical to the function of the fusion protein, provided that the subdomain linker peptide permits a functional connection between the subdomains. Amino acid sequences which may be usefully employed as linkers include those disclosed in Maratea et al., Gene 40:39-46 (1985), Murphy et al., Proc. Nat. Acad Sci USA, 83:8258-8562 (1986), U.S. Pat. Nos. 4,935,233 and 4,751,180, all of which are incorporated by reference.

The term “functional connection” in the context of a linker peptide indicates a connection that facilitates folding of the polypeptides of each domain into a three-dimensional structure that allows the linked fusion polypeptide to mimic some or all of the functional aspects or biological activities of the domain from which it is derived. The term functional connection also indicates that the linked domains possess at least a minimal degree of stability, flexibility and/or tension that would be required for the binding domain to function as desired. In one embodiment of the present invention, the domain linker peptides comprise or consist of the same amino acids. In another embodiment, the amino acids of the domain linker peptides are different from one another.

In additional embodiments of the present invention, at least one polynucleotide codes for a fusion protein with more than two domains, with at least one of the multiple domains coding for a domain that facilitates detection of the transgenic cells that express the protein of interest. In specific embodiments, the polynucleotide that codes for the fusion protein with multiple domains comprises at least one codon that codes for the nnAA. In specific embodiments, the polynucleotide that codes for the fusion protein with multiple domains comprises at least one codon that codes for at least one nnAA in between each domain. In this embodiment, the multiple codons that code for the nnAA may or may not code for the same nnAA. Thus, if the transgenic cell comprises more than one set of orthogonal tRNA/orthogonal tRNA synthetase, i.e., each set corresponds to a different nnAA, it would be possible to include a specific nnAA in the culture medium, but not the other. In this situation, one could control the production of a protein that has one or two, or more, domains fused to the first domain.

The polynucleotide encoding the fusion protein should code at least one protein of interest. The methods of the present invention are not limited by the identity of the protein of interest. Methods of generating expression vectors that include polynucleotides coding for fusion proteins are so well-known in the art that virtually any coding sequence can be used to generate any protein of interest. Examples of proteins of interest include structural proteins, enzymes, antibodies and other defense proteins, signaling proteins, regulatory proteins, transport proteins, sensory proteins, motor proteins and storage proteins.

The polynucleotide encoding the fusion protein should also code for at least a second domain that facilitates detection of the transgenic cells that express the protein of interest. In one embodiment, the second domain is an anchor domain that anchors the entire fusion protein to the cell membrane of the transgenic cell expressing the fusion protein. As used herein, an anchor domain is a domain that permits the protein of interest of interest to be “displayed” on the surface of the transgenic cell, such that the protein of interest is generated within the cell but the cell cannot secrete the protein of interest into the cell culture environment separate from the cell. The anchor domain need not be a complete protein on its own and included functional parts of protein able to anchor the fusion protein to the cell membrane of the transgenic cell expressing the fusion protein. For example, the transmembrane domain portion of a more complex protein can be used as the anchor domain in the methods of the present invention. Examples of anchor domains include but are not limited to a single pass transmembrane domain, a transmembrane beta-barrel or any portion thereof. Specific examples of transmembrane domains that can be used as the anchor domains of the present invention include, but are not limited to, a transmembrane domain from a member of the tumor necrosis factor receptor superfamily, CD30, platelet derived growth factor receptor (PDGFR, e.g. amino acids 514-562 of human PDGFR; Chestnut et al. 1996 J Immunological Methods 193:17-27; also see Gronwald et al. 1988 PNAS 85:3435); nerve growth factor receptor, Murine B7-1 (Freeman et al. 1991 J Exp Med 174:625-631), asialoglycoprotein receptor H1 subunit (ASGPR; Speiss et al. 1985 J Biol Chem 260:1979-1982), CD27, CD40, CD120a, CD120b, CD80 (Freeman et al. 1989 J Immunol 143:2714-22) lymphotoxin beta receptor, galactosyltransferase (E.G. GenBank accession number AF155582), sialyly transferase (E.G. GenBank accession number NM-003032), aspartyl transferase 1 (Asp1; e.g. GenBank accession number AF200342), aspartyl transferase 2 (Asp2; e.g. GenBank accession number NM-012104), syntaxin 6 (e.g. GenBank accession number NM-005819), ubiquitin, dopamine receptor, insulin B chain, acetylglucosaminyl transferase (e.g. GenBank accession number NM-002406), APP (e.g. GenBank accession number A33292), a G-protein coupled receptor, thrombomodulin (Suzuki et al. 1987 EMBO J 6, 1891), A-agglutinin-binding subunit, and TRAIL receptor. Examples of transmembrane domains are also described in PCT Publication Nos. WO 1998/021232, WO 2003/104415, and WO 2007/047578.

In other embodiments, the anchor domain is a glycosylphosphatidylinositol (GPI) signal peptide that promotes anchoring of the protein of interest to a GPI moiety present in the cell membrane. GPI signal peptides are well-known in the art and include the signal peptides from the GPI-anchored proteins discussed in Chapter 11 of Essentials of Glycobiology, 2nd. Edition, Varki, A., et al. Eds., Cold Spring Harbor Laboratory Press (2009), which is incorporated by reference. Specific examples of GPI signal peptides include the GPI signal peptide from the decay accelerating factor 7 (DAF-7), nogo receptor, trail decoy receptor, folate receptor, membrane anchored serine proteases, and scrapie prion protein.

In one embodiment, the second domain is a tag that labels the protein of interest. A tag can be any detectable molecule, for example a peptide sequence, attached to a protein to facilitate detection or purification of an expressed protein. A fusion protein of the current invention may comprise two, three, four or more domains, each of which could be a distinct tag. If the fusion protein of the current invention contains more than one tag, the tag may or may not be chemically identical. In certain embodiments, a tag can be an affinity, epitope, or fluorescent tag. Affinity, epitope, or fluorescent tags are recognized in the art. Examples of affinity tags that are part of fusion proteins of the current invention include, but are not limited to, glutathione-S transferase (GST), poly-histidine tag (His), calmodulin binding protein (CBP), and maltose-binding protein (MBP). Examples of epitope tags that are part of fusion proteins of the current invention include, but are not limited to, myc, human influenza hemagglutinin (HA), and FLAG. Examples of fluorescent tags that are part of fusion proteins of the current invention include, but are not limited to, green fluorescent proteins (GFP, AcGFP, ZsGreen), red-shifted GFP (rs-GFP), red fluorescent proteins (RFP, including DsRed2, HcRed1, dsRed-Express), yellow fluorescent proteins (YFP, Zsyellow), cyan fluorescent proteins (CFP, AmCyan), a blue fluorescent protein (BFP) and the phycobiliproteins, as well as the enhanced versions and mutations of these proteins. For some fluorescent proteins enhancement indicates optimization of emission by increasing the proteins' brightness or by creating proteins that have faster chromophore maturation. These enhancements can be achieved through engineering mutations into the fluorescent proteins.

The methods comprise culturing the transgenic cells in culture conditions that permit protein synthesis. The transgenic cells being cultured according to the present invention can be cultured and plated or suspended according to the experimental conditions as needed by the technician. The examples herein demonstrate at least one functional set of culture conditions that can be used in conjunction with the methods described herein. If not known, plating or suspension and culture conditions for promoting protein synthesis for a given cell type can be determined by one of ordinary skill in the art using only routine experimentation. Cells may or may not be plated onto the surface of culture vessels, and, if plated, attachment factors can be used to plate the cells onto the surface of culture vessels. If attachment factors are used, the culture vessels can be pre-coated with a natural, recombinant or synthetic attachment factor or factors or peptide fragments thereof, such as but not limited to collagen, fibronectin and natural or synthetic fragments thereof.

The cell seeding densities for the culture condition can be manipulated for the specific culture conditions needed. For routine culture in plastic culture vessels, a seeding density of the transgenic cells can be from about 1×10⁴ to about 1×10⁷ cells per cm², which is fairly typical, e.g., 1×10⁶ cells are often cultured in a 35 mm²-100 mm² tissue culture petri dish. Cell density can be altered as needed at any passage.

If one were culturing mammalian transgenic cells for protein synthesis using the methods of the present invention, these cells are typically cultivated in a cell incubator at about 37° C. at normal atmospheric pressure. The incubator atmosphere is normally humidified and often contain about 3-10% carbon dioxide in air. Temperature, pressure and CO₂ concentration can be altered as necessary, provided the cells are still viable. Culture medium pH can be in the range of about 7.1 to about 7.6, in particular from about 7.1 to about 7.4, and even more particular from about 7.1 to about 7.3.

The transgenic cells are cultured under conditions to permit protein synthesis to occur. When one or more nnAAs are present during the culture conditions, the full-length fusion protein, comprising the two or more domains, will be synthesized and the protein of interest will be either anchored to or on the transgenic cell or the protein of interest will be labeled. When one or more nnAAs are not present during the culture conditions, the full-length fusion protein will not be synthesized. Instead, only the protein of interest is expressed, since the codon coding for the nnAA will act as a stop codon when no nnAAs are present. In specific embodiments, the nnAA is placed in cell culture conditions with the transgenic cells for about 48 hours or less, after which the cell culture medium containing the nnAAs is replaced with cell culture medium without nnAAs. Removing cell culture medium and washing the cells with buffers, such as but not limited to PBS, to remove trace amounts of the previous cell culture medium are routine. In more specific embodiments, the nnAA is placed in cell culture conditions with the transgenic cells for less than about 48 hours, less than about 46 hours, less than about 44 hours, less than about 42 hours, less than about 40 hours, less than about 38 hours, less than about 36 hours, less than about 34 hours, less than about 32 hours, less than about 30 hours, less than about 28 hours, less than about 26 hours, less than about 24 hours, less than about 22 hours, less than about 20 hours, less than about 18 hours, less than about 16 hours, less than about 14 hours, less than about 12 hours, less than about 10 hours, less than about 8 hours, less than about 6 hours, less than about 5 hours, less than about 4 hours, less than about 3 hours, less than about 2 hours, less than about 1 hour, after which the cell culture medium containing the nnAAs is replaced with cell culture medium without nnAAs.

In other embodiments, the concentration of the one or more nnAA in the cell culture medium can vary. In one specific embodiment, the total concentration of the one or more nnAA in the cell culture medium is about 5 mM or less. In a more specific embodiment, the total concentration of the one or more nnAA in the cell culture medium is between about 2 mM to about 10 μM. In even more specific embodiments, the total concentration of the one or more nnAA in the cell culture medium is between about 10 μM to about 20 μM, about 20 μM to about 30 μM, about 30 μM to about 40 μM, about 40 μM to about 50 μM, about 50 μM to about 60 μM, about 60 μM to about 70 μM, about 70 μM to about 80 μM, about 80 μM to about 90 μM, about 90 μM to about 100 μM, about 100 μM to about 120 μM, about 120 μM to about 140 μM, about 140 μM to about 160 μM, about 160 μM to about 180 μM, about 180 μM to about 200 μM, about 200 μM to about 250 μM, about 250 μM to about 300 μM, about 300 μM to about 350 μM, about 350 μM to about 400 μM, about 400 μM to about 450 μM or about 450 μM to about 500 μM, about 500 μM to about 550 μM, about 550 μM to about 600 μM, about 600 μM to about 650 μM or about 650 μM to about 700 μM, about 700 μM to about 750 μM, about 750 μM to about 800 μM, about 800 μM to about 850 μM or about 850 μM to about 900 μM, about 900 μM to about 950 μM, about 950 μM to about 1.0 mM, about 1.0 mM to about 1.1 mM, about 1.1 mM to about 1.2 mM, about 1.2 mM to about 1.3 mM, about 1.3 mM to about 1.4 mM, about 1.4 mM to about 1.5 mM, about 1.5 mM to about 1.6 mM, about 1.6 mM to about 1.7 mM, about 1.7 mM to about 1.8 mM, about 1.8 mM to about 1.9 mM, about 1.9 mM to about 2.0 mM, about 2.0 mM to about 2.1 mM, about 2.1 mM to about 2.2 mM, about 2.2 mM to about 2.3 mM, about 2.3 mM to about 2.4 mM, about 2.4 mM to about 2.5 mM, about 2.5 mM to about 2.6 mM, about 2.6 mM to about 2.7 mM, about 2.7 mM to about 2.8 mM, about 2.8 mM to about 2.9 mM, about 2.9 mM to about 3.0 mM, about 3.0 mM to about 3.1 mM, about 3.1 mM to about 3.2 mM, about 3.2 mM to about 3.3 mM, about 3.3 mM to about 3.4 mM, about 3.4 mM to about 3.5 mM, about 3.5 mM to about 3.6 mM, about 3.6 mM to about 3.7 mM, about 3.7 mM to about 3.8 mM, about 3.8 mM to about 3.9 mM, about 3.9 mM to about 4.0 mM; about 4.0 mM to about 4.1 mM, about 4.1 mM to about 4.2 mM, about 4.2 mM to about 4.3 mM, about 4.3 mM to about 4.4 mM, about 4.4 mM to about 4.5 mM, about 4.5 mM to about 4.6 mM, about 4.6 mM to about 4.7 mM, about 4.7 mM to about 4.8 mM, about 4.8 mM to about 4.9 mM, about 4.9 mM to about 5.0 mM.

If the fusion protein is synthesized, i.e., the culture conditions include one or more nnAAs, the protein of interest will be displayed on the surface of the transgenic cell. The methods of the present invention will then include methods for determining the level or amount of protein of interest of interest produced in the transgenic cells. By determining the level of protein of interest that is displayed on the individual cell or within a subgroup of cells within the population of transgenic cells, one can then determine which individual cells or subgroups of cells produce a higher quantity of the protein of interest of interest compared to the remaining cells of the population of transgenic cells.

Methods of determining and quantifying proteins that are displayed on the surface of cells are routine in the art. In one embodiment, determining levels of the protein of interest that are displayed on the surface of the transgenic cells comprises flow cytometry. In other embodiments, determining levels of the protein of interest that are displayed on the surface of the transgenic cells comprises cell-based ELISA, homogeneous assays, Western blots, ligand binding, antigen binding, functional assays, antibody-dependent killing assays.

In one specific embodiment, the transgenic cells that produce lesser amounts of the protein of interest that are displayed on the surface of the transgenic cells are separated from the remainder of the population of transgenic cells. In one specific embodiment, the transgenic cells that produce higher amounts of the protein of interest that are displayed on the surface of the transgenic cells are separated from the remainder of the population of transgenic cells. Methods of separating cells from within a population of cells are routine in the art and include but are not limited to fluorescence-activated cell sorting (FACS), bead based sorting, ClonePix, Berkley lights technology, and limited-dilution cloning and expression assessment.

As used herein, when determining the level, quantity or amount of the protein of interest, these determinations can be relative or absolute. For example, when using FACS to sort the higher producers from the remaining population of cells, one determination could simply be measuring a brighter or more intense fluorescent signal relative to the other members of the population of cells.

As used herein, the term “higher” when used in conjunction with quantities of protein of interest is a relative term that can be set by the technician. For example, if using fluorescence as a surrogate measurement of the quantity of protein of interest displayed on the surface of the transgenic cells, the operator can set a minimal amount of fluorescence that must be displayed to be considered a “higher” producer. In the alternative, the technician can simply select a portion or percentage of cells that display the highest level of protein of interest. In specific embodiments, the term “higher,” when used in conjunction with quantities of protein of interest, means about the top 50%, about the top 45%, about the top 40%, about the top 35%, about the top 30%, about the top 25%, about the top 20%, about the top 15%, about the top 10%, about the top 5% or about the top 1% of cells that display the protein of interest from the initial population of transgenic cells that are placed in conditions to permit synthesis of the fusion protein.

As used herein, the term “lesser” when used in conjunction with quantities of protein of interest is a relative term that can be set by the technician. For example, if using fluorescence as a surrogate measurement of the quantity of protein of interest displayed on the surface of the transgenic cells, the operator can set a minimal amount of fluorescence that must be displayed to not be considered a “lesser” producer. In the alternative, the technician can simply select a portion or percentage of cells that display the lowest level of protein of interest. In specific embodiments, the term “lesser,” when used in conjunction with quantities of protein of interest, means about the bottom 50%, about the bottom 45%, about the bottom 40%, about the bottom 35%, about the bottom 30%, about the bottom 25%, about the bottom 20%, about the bottom 15%, about the bottom 10%, about the bottom 5% or about the bottom 1% of cells that display the protein of interest of interest from the initial population of transgenic cells that are placed in conditions to permit synthesis of the fusion protein.

Once the cells are sorted and thus isolated from the initial population of transgenic cells to either higher producers or “not lesser producers” of the protein of interest of interest, these isolated cells can then be placed in a subsequent cell culture environment. The subsequent cell culture environment may or may not contain nnAAs. In one embodiment, the subsequent cell culture environment does not contain nnAAs such that the protein of interest is produced, but not as part of a fusion construct. In another embodiment, the subsequent cell culture environment initially does not contain nnAAs, but, after a period of time or after passaging the cells, the one or more nnAAs are added the cell culture environment. Once the nnAAs are added, the cells can be permitted to generate the fusion protein and this subsequent population of transgenic cells can be re-evaluated for levels of production. This repeating of the inclusion of the nnAAs into the cell culture medium and subsequent determining of protein levels can be used to further isolate the higher producers of the protein of interest of interest.

Once the cells are placed in a cell culture environment that permit protein synthesis and without any nnAAs, the cells can then produce the first domain of the fusion protein. Thus, for example, an un-anchored protein may then be secreted into the cell culture medium and subsequently isolated using traditional protein isolation techniques. Likewise, an un-labeled protein, which is correctly folded, can be generated in the transgenic cell.

The examples herein are meant to be illustrative and are not intended to limit the scope of the invention.

### EXAMPLES

### Example 1—Complex Membrane Protein Expression

A plasmid encoding the pyrrolysyl tRNA synthetase (pylRS) and tRNApyl of Methanosarcina mazei was generated to facilitate incorporation of non-natural lysine derivatives at amber stop codons. This tRNA synthetase and tRNA pair is orthogonal in a variety of host cells and can accommodate a variety of lysine analogs without modification. The FLAG-tagged pylRS and tRNApyl from pMOAV2 was first transferred to pDONR221 and finally to pEF-DEST51-Puro via the Gateway BP Clonase reaction to generate pEF-DEST51-Puro-MOAV2. Within this vector, the cytomegalovirus (CMV) promoter controls transcription of the modified pylRS gene and the U6 snRNA promoter controls transcription of 18 copies of the tRNApyl gene.

Plasmids encoding complex membrane proteins were generated as fusion proteins of enhanced green fluorescence protein (eGFP) separated by either an amber stop codon (TAG) or a lysine codon (AAG) in the lentiviral vector (pCDH1-CMV-MCS-Puro, System Biosciences, Palo Alto, Calif.) which had been modified to replace the EF1-puromycin resistance cassette with that of the SV40-blasticidin resistance cassette. The lysine-containing variant served as a ‘read-through’ control protein in cell lines. Expression vectors were further modified to facilitate detection of untagged and tagged proteins by Western blot: (1) prior to the amber stop/lysine codon fusion protein junction, membrane protein sequences were modified to contain the AVITAG™ sequence (GLNDIFEAQKIEWHE) and the Rho 1D4 epitope (TETSQVAPA-COOH) separated by triple alanine (AAA) linkers; and, (2) after the amber stop/lysine junction, eGFP was preceded by a glycine/serine rich linker (G(GSG)₄G) and followed by a glycine/serine rich linker (G(GSG)₄G), FLAG epitope (DYDDDDK), a glycine/serine linker (GSG) and Rho 1D4 epitope. The Rho 1D4 antibody is specific to the epitope at the protein's C-terminus; therefore, internal epitopes, as is the case during amber suppression, are not detected. DNA sequencing was used to confirm all constructs.

Jump-In CHOK1 (Invitrogen, Carlsbad, Calif.) were maintained and propagated in F12 medium. Medium was supplemented with supplemented with 10% FBS and 1 mM 5-(((allyloxy)carbonyl)amino)pentanoic acid, a non-hydrolyzable pyrrolysine analog that interferes with pylRS function and promotes healthy growth of cell lines containing pylRS and tRNApyl. Jump-In CHOK1 were stably transfected with pEF-DEST51-Puro-MOAV2 and selected with 5 μg/mL puromycin (Invitrogen) beginning 24 h post-transfection. To assess amber suppression in cell lines, puromycin-selected outgrowth consisting of a selected, mixed population were transiently transfected with pMax-mCherryOpt-GFP amb encoding an mCherry_{AMB}GFP reporter under control of the CMV promoter and grown in 2 mM N6-((2-azidoethoxy)carbonyl)-L-lysine hydrochloride (lysine azide) (IRIS Biotech, Marktredwitz, Germany). With this reporter construct, transfected cells exhibit mCherry fluorescence and cells suppressing the amber codon exhibit mCherry and GFP fluorescence. Cells were assessed by flow cytometry and fluorescence-activated cell sorting on a BD FACSAria III for fluorescence in the PE-Texas Red (mCherry) and FITC (GFP) channels using 100 μm nozzle and PBS as sheath fluid. Cells exhibiting mCherry and GFP fluorescence were sorted into 96-well plates containing 200 μL growth media supplemented with 1 mM decoy and 5 μm/mL puromycin. Following expansion in 96-well plates, 12 colonies were further expanded into 12-well plates and finally T75 flasks before being frozen and stored in liquid nitrogen. Cell lines were renamed ‘Jump-In CHOK1+MOAV2’.

To assess the specificity of amber suppression in individual Jump-In CHOK1+MOAV2 Clones 1-12, the mCherry_{AMB}GFP reporter plasmid was transiently transfected and clones were grown in either media supplemented with 1 mM decoy or 2 mM lysine azide. Transfected cells were analyzed on a LSR Fortessa for fluorescence in the PE-Texas Red (mCherry) and FITC (GFP) channels. From these, Clone 7 was selected for characterization of the inducible system.

Lentiviruses for expression of membrane proteins were generated by transient transfection of DNA into suspension 293F cells as follows: 1.65 μg lentiviral vector plasmid encoding membrane protein and 8.35 μg pPACKH1 (System Biosciences, Palo Alto, Calif.) were transfected using 293Fectin (Life Technologies Carlsbad, Calif.). Cellular supernatants containing lentivirus were harvested two days post-transfection and concentrated 50-fold by ultracentrifugation. Clone 7 cells were spin-transduced with lentivirus (MOI=2.5) and 2 μm/mL polybrene (Millipore Sigma, Milwaukee, Wis.) in 2 mL F12 supplemented with 10% FBS and 1 mM decoy, followed by centrifugation at 2,500 rpm for 1.5 h at 32° C. Polybrene was removed from cells 24-h post-transduction and cells were selected for blasticidin resistance (10 μg/mL) two days post-transduction. The blasticidin selection was complete after approximately 7-10 days; cells that survived the antibiotic selection represent a selected, mixed population that varies in genetic loci, copy number and expression phenotype.

For cell sorting of membrane protein cell lines, Clone 7 cells expressing EphA2, Claudin 1, CXCR2, or CXCR4 were washed extensively with PBS and grown in F12 supplemented with 10% FBS and 2 mM lysine azide to induce translation of the GFP tag for 48 h prior to cell sorting. On the day of cell sorting, cells were detached with Tryp-LE (Invitrogen), washed and resuspended in FACS Buffer (PBS, pH 7.4 supplemented with 2% FBS), and filtered. Samples were run on a BD INFLUX™ Cell Sorter (BD Biosciences, San Jose, Calif.), where the top 10% of GFP positive cells were collected and expanded in bulk.

For flow cytometry (FCM), cells were detached from culture dishes with Tryp-LE, washed with FACS Buffer, and stained with 10 μg/mL Hy29-1 (CXCR2), X2-753 (CXCR2), or MEDI3185 (CXCR4) in 100 μL FACS Buffer for 30 min on ice. Cells were washed extensively, stained with 10 μm/mL goat anti-human IgG (H+L)-Alexa Fluor 647 (Thermo Fisher Scientific) in FACS Buffer for 30 min on ice and washed extensively. The isotype control antibody, R347, was used as a negative control. Samples were run on a MACSQuant VYB (Miltenyi Biotec, Auburn, Calif.) and gated for live cells. Data analysis was performed using FlowJo software (Tree Star, Inc., Ashland, Oreg.).

Having confirmed that this inducible approach could be used to control this switchable tag system, tag-based enrichment of cell lines by FACS was investigated to determine if it could be used as an alternative to protein-specific antibody methods. The rationale for this approach derives from the fixed ratio of membrane protein:eGFP within tagged fusion protein constructs. The selected, mixed population cell lines expressing the ‘AMB’ versions of the model membrane proteins were exposed to cell sorting following 48 h lysine azide induction. The top 10% of GFP positive cells were isolated and expanded in bulk. Alternatively, sorted cells could have been cloned by single cell dilution; however, the aim of this approach was to evaluate the inducible method for membrane protein enrichment, so bulk expansion was selected to simplify workflows. To assess cell sorting enrichment, selected, mixed and sorted cell populations expressing untagged model membrane proteins were evaluated by anti-Rho 1D4 and tubulin Western blot and membrane protein-specific FCM.

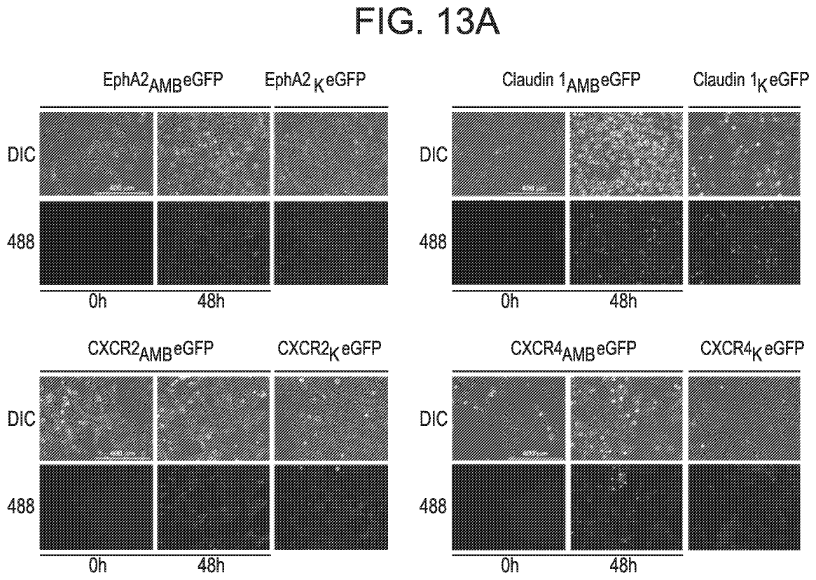

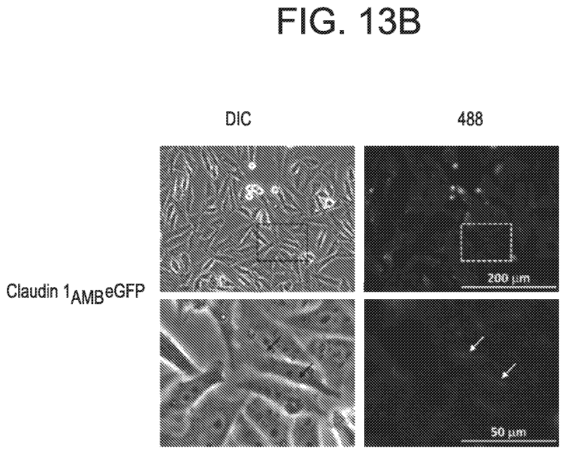

To assess whether cell sorting led to an overall enrichment in high expression cells, total expression of model membrane proteins was evaluated by Western blot using the anti-Rho 1D4 antibody. It was found that EphA2, Claudin 1, CXCR2 and CXCR4 showed ˜2-, 1.3, 7.6- and 1.5-fold expression improvements over mixed populations, respectively (FIG. 13A). These results suggest that sites within biological membranes may be limiting as the same levels of CXCR2 enrichment was not observed for the well-expressed model membrane proteins, EphA2, Claudin 1 and CXCR4. These results show, however that this approach is most helpful for complex proteins with low expression levels.

Because Western blot provides an assessment of total expression levels irrespective of protein localization, protein-specific FCM was pursued to assess surface expression of the model membrane proteins. CXCR2 and CXCR4 were examined due to the levels of enrichment observed by Western blot and due to the relevance of receptor surface expression on functional screens and drug discovery. To achieve this end, the anti-CXCR2 antibodies, HY29-1 and X2-753, and the anti-CXCR4 antibody, MEDI3185 were used. HY29-1 and X2-753 possess distinct CXCR2 epitopes with variable ligand inhibition in functional assays. Using HY29-1 and X2-753 on sorted and selected, mixed populations expressing ‘untagged’ CXCR2, a 2.25- and 2.53-fold improvement in expression was observed for the sorted over the selected, mixed populations, respectively (FIG. 13B). Then, MEDI3185 was used for FCM analysis of CXCR4 expressing cells, where MEDI3185 is a potent anti-CXCR4 antagonistic antibody. Using MEDI3185 on sorted and selected, mixed populations expressing ‘untagged’ CXCR4, a roughly 10% improvement was observed in surface expression (FIG. 13).

The differences observed for total and surface expression enrichments of CXCR2 and CXCR4 as determined by Western blot and FCM analyses, respectively, highlight the differences in organellar location of these enrichments. For example, enrichments to total expression are expected to occupy all biological membranes of the secretory pathway, including the endoplasmic reticulum, the Golgi apparatus, and the plasma membrane. For CXCR2, the 7.6-fold enrichment of total expression resulted in ˜2.4 enrichment in surface expression. Although in some applications, such as purification of membrane proteins for biophysical and structural characterization, total expression enrichment may be sufficient; however, for other applications, such as cell-based drug discovery and functional assays, enrichments to surface expression are desired. The desired expression phenotype may be achieved through multiple rounds of bulk sorting, single cell sorting or use of a fusion strategy specific to detection of surface expression.

Whole-cell lysates from Clone 7 control cells and 0, 24, or 48 h post-induction of CMP-containing cells were generated by lysing 1E7 cells in 200 μL PBS containing 1% sodium dodecyl sulfate (SDS) and 1× complete protease inhibitors (Roche) followed by resuspension in 1× Sample Buffer (Life Technologies) and heating at 55° C. for 10 min. Proteins were then separated by 4-12% sodium dodecyl sulphate-polyacrylamide gel electrophoresis (SDS-PAGE) and transferred to nitrocellulose membranes (Thermo Scientific, Kirya Shmona, Israel). Blots were blocked with STARTINGBLOCK™ (PBS) Blocking Buffer (Thermo Scientific, Rockford, Ill.) for 1 h and probed with primary antibodies against Rho 1D4 (1:1000, ab5417, Abcam, Cambridge, Mass.), FLAG® M2-HRP conjugated (1:5000, A8592, Sigma, St. Louis, Mo.), GFP (1:5000, ab6556, Abcam), or Tubulin (1:5000, ab6160, Abcam) overnight at 4° C. Blots were washed extensively and incubated with the appropriate horseradish peroxidase (HRP)-conjugated secondary antibodies for 1 h at room temperature. Signals were detected using LumiGLO chemiluminescent substrates A+B (SeraCare, Milford, Mass.) and recorded on film. A qualitative estimate of expression improvements was achieved by comparison of band intensities using ImageJ software. Raw data was normalized against the tubulin loading control. Western blot images were uniformly modified for brightness and contrast.

Fluorescence imaging of control cells and 0, 24, or 48 h post-induction of AMB membrane protein-expressing cell lines were digitally captured with 10× or 20× optical objectives using an EVOS™ FL Auto Imaging System (Life Technologies, Carlsbad, Calif.). Representative images from multiple experiments were uniformly adjusted for brightness and contrast.

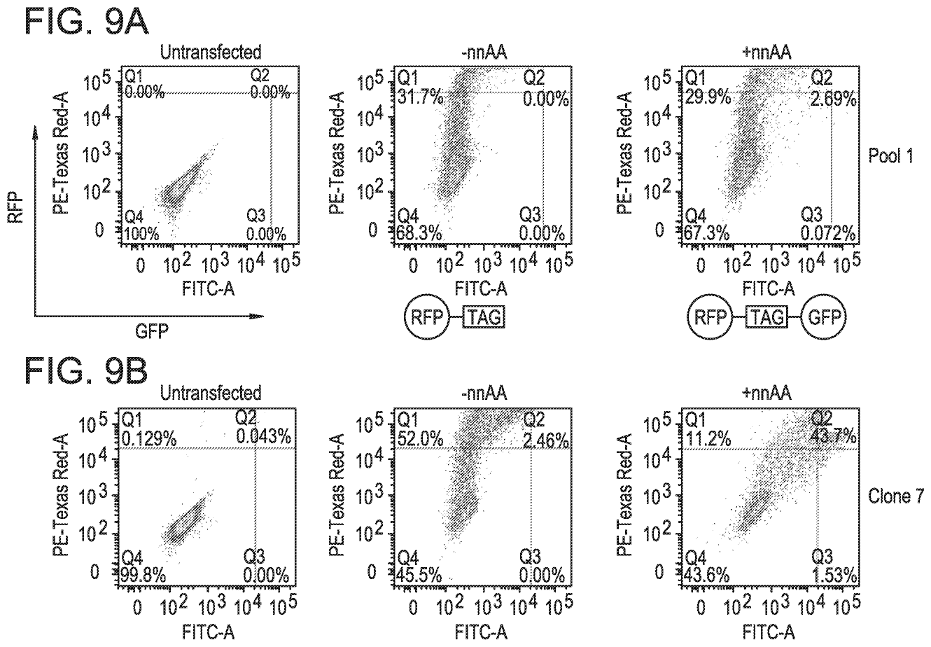

To investigate the utility of inducible approach, a clonal cell line capable of homogeneous, high efficiency amber suppression was generated. Jump-In CHOK1 cells were transfected to stably express the pyrrolysyl tRNA synthetase (pylRS) and tRNA (CUA pyl) genes from Methanosarcina mazei. The Jump-In CHOK1 cell line was selected as a model system due to: 1) the facile generation of high-expressing cell pools for drug discovery and 2) reports that CHO-derived cell lines support efficient amber suppression. Selected cells were assessed for their amber suppression potential by transient transfection of a reporter plasmid encoding mCherryAMBGFP that contains an amber codon interrupting the fusion protein. Thus, the construct enables constitutive expression of mCherry-GFP fusion. The efficiency of amber suppression within a cell population can be gauged by the percentage of cells expressing the fusion protein (mCherry+/GFP+). Using this reporter, efficient amber suppression was rare within the heterogeneous population, where only ˜2% of cells showed efficient amber suppression (FIG. 9A) in response to media supplemented with the non-natural amino acid N6-((2-azidoethoxy)carbonyl)-L-lysine (lysine azide). Cells exhibiting the desired phenotype (top 2% mCherry and GFP positive) were isolated by single cell sorting and expanded. One of the isolated clones (Clone 7) was selected for further characterization as this clone exhibited high amber suppression activity (43%) with media supplemented with lysine azide and low background suppression (2.5%) in the absence of lysine azide (FIG. 9B).

To evaluate whether this approach could be applied to the expression of transmembrane proteins, Clone 7 from above was transduced with lentivirus encoding model membrane proteins that differed in transmembrane domain complexity, N- and C-terminal orientation, physiological function, and expression level (Table 1), below.

TABLE 1
Diversity of model membrane proteins tested

| | Number of | N- | C- | | | | |
|---|---|---|---|---|---|---|---|
| Membrane | Transmembrane | terminus | terminus | Expression | Physiological | Detection | |
| Protein | Domains | Orientation | Orientation | Level | Function | Reagent(s) | References |
| Ephrin | 1 | Extracellular | Intracellular | +++ | Receptor | B233 and | Dall'Acqua, W. F., et al., |
| type-A | | | | | | 1C1 | Methods, 2005. |
| receptor 2 | | | | | | | 36(1): p. 43-60; |
| (Epha2) | | | | | | | Jackson, D., et al., |
| | | | | | | | Cancer Res, 2008. |
| | | | | | | | 68(22): p. 9367-74 |
| Claudin 1 | 4 | Intracellular | Intracellular | +++ | Tight | ab115783 | Bonander, N., et al., |
| | | | | | Junction | | PLoS One, 2013. |
| | | | | | | | 8(5): p. e64517 |
| C-X-C | 7 | Extracellular | Intracellular | +/− | GPCR | Hy-29-1 | Boshuizen, R. S., et al., |
| chemokine | | | | | | and X2- | MAbs. 6(6): p. 1415-24; |
| receptor | | | | | | 753 | R ossant, C. J., et al., |
| (CXCR2) | | | | | | | MAbs. 6(6): p. 1425-38 |
| C-X-C | 7 | Extracellular | Intracellular | + | GPCR | MEDI3185 | Peng, L., et al., |
| chemokine | | | | | | | MAbs. 8(1): p. 163-75; |
| receptor 4 | | | | | | | Schwickart, M., et al., |
| (CXCR4) | | | | | | | Cytometry B Clin Cytom. |
| | | | | | | | 90(2): p. 209-19 |

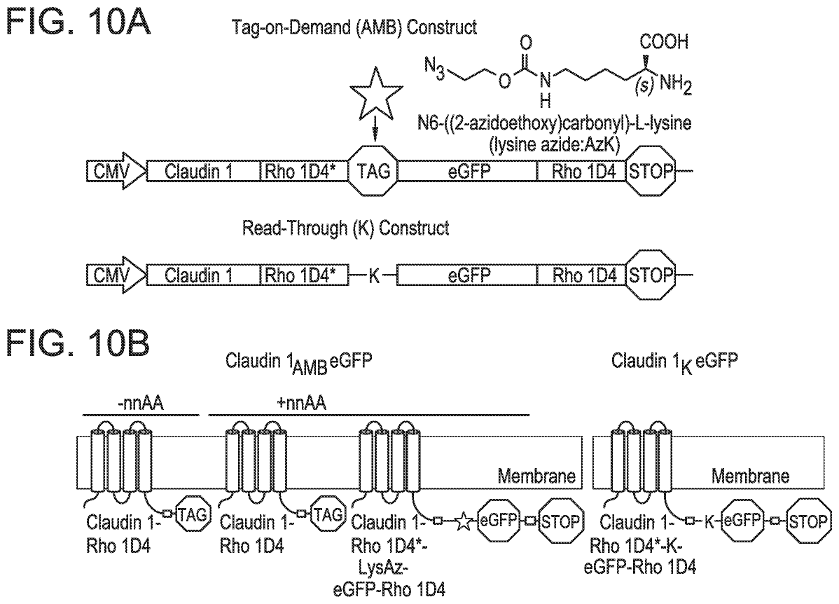

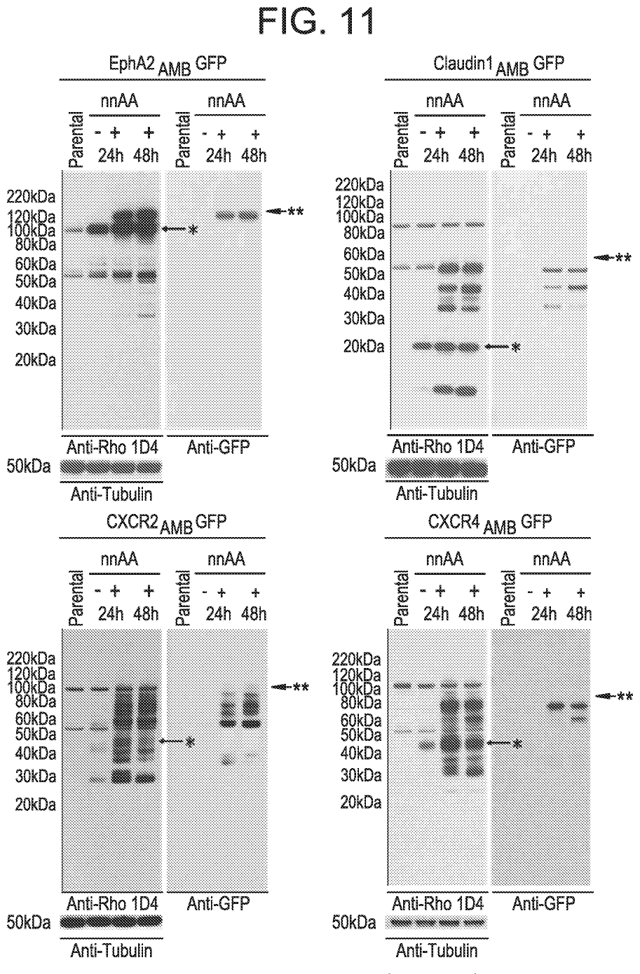

Ephrin type-A receptor 2 (EphA2), Claudin 1, C-X-C chemokine receptor 2 (CXCR2) and C-X-C chemokine receptor 4 (CXCR4) were expressed as amber suppression-dependent and read-through fusion proteins with enhanced green fluorescent protein (eGFP) carrying the FLAG and Rho 1D4 epitopes (FIG. 10). Native membrane protein sequences were further modified to contain the Rho 1D4 epitope prior to the amber stop codon to enable comparative analysis of expression. To evaluate this inducible concept for ‘switchable’ expression, cells expressing model membrane proteins were analyzed by Western blot (FIG. 11) and fluorescence microscopy (FIG. 13A) after nnAA induction. Amber suppression was tightly controlled for all proteins tested. Tagged proteins were detected only when cell culture mediums were supplemented with lysine azide. Furthermore, EphA2 and Claudin 1 expressed well in this system, but CXCR2 and CXCR4 expressed poorly, suggesting that expression may not be a critical obstacle for all proteins. Tagged EphA2, CXCR2, and CXCR4 displayed well-distributed localization in cellular membranes, however, tagged Claudin 1 showed specific localization to cell-to-cell contacts consistent with its role in the formation of tight junctions (FIG. 13B). In all model proteins tested, cells expressing the tagged variants of membrane proteins showed similar cellular distributions to the corresponding read-through variants.

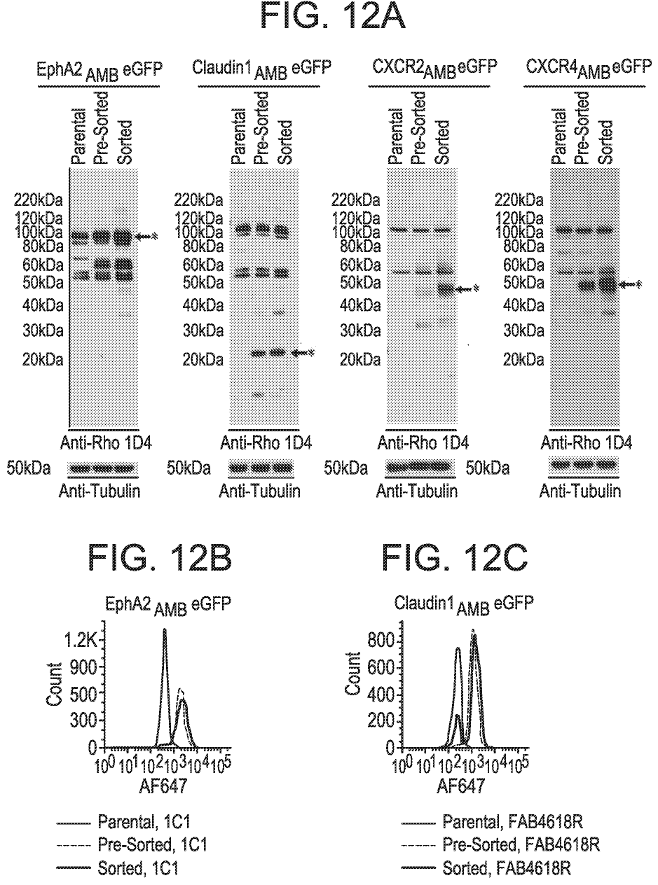

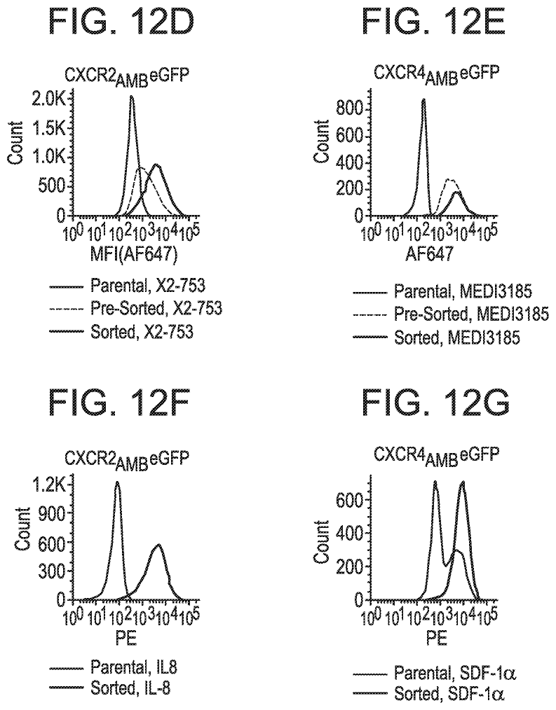

Having confirmed this approach can be used to control ‘switchable’ expression of model membrane proteins, we then investigated whether tag-based enrichment of cell lines by FACS could be used as an alternative to protein-specific antibody methods. Following 48 hours of induction, the top 10% of GFP-positive cells expressing the model membrane proteins were sorted and expanded. The enrichment of cell populations expressing improved levels of ‘untagged’ membrane proteins was evaluated by Western blot and cell surface FCM. As shown in FIG. 12A, sorted EphA2, Claudin 1, CXCR2 and CXCR4 cell lines exhibited ˜200-800% improvement in total expression over pre-sorted populations as determined by Western blot. However, this improvement in total expression does not necessarily reflect a concomitant increase in cell surface expression. To determine the specific improvement in surface expression levels, cells were labeled using reference antibodies specific for each of the model membrane proteins and the fluorescence levels were quantified by FCM. Interestingly, the expression-limited G protein-coupled receptor CXCR2 showed a ˜2.5-fold increase in surface staining over the pre-sorted population. In contrast, the surface expression of sorted EphA2, Claudin 1 and CXCR4 cells was only marginally improved, at 10-20% (FIGS. 12B-E), which suggests that these proteins are already at or near their maximal surface expression levels in these cells. Finally, the structural integrity of the receptors in enriched cell lines was tested by their ability to bind their natural ligand. We focused these efforts on CXCR2 and CXCR4 due to the complexity of GPCR folding [40]. Streptavidin complexes carrying biotinylated interleukin 8 (IL-8) or stromal-cell derived factor-1α (SDF-1α) ligands were exposed to parental and sorted cell lines expressing ‘untagged’ CXCR2 or CXCR4, respectively, and ligand binding was assessed by FCM. Despite low-level endogenous expression of CXCR2 and CXCR4 in parental cells, the high-expressing CXCR2 and CXCR4 cell lines exhibited specific staining, indicating proper folding of the receptors (FIGS. 12F and G). Collectively, these results validate this approach as a platform and indicate that is may be particularly beneficial for enrichment and selection of cell lines expressing membrane proteins with low starting expression levels.

### Example 2—Antibody Expression

Genes encoding a heavy chain (HC) and light chain (LC) of an IgG directed to EphA2 were placed under control of CMV promoters near UCOE elements in the vector pCLD (IgG control). Membrane associated IgGs were made by expressing the HC fused to the glycosylphosphatidylinositol membrane anchor sequence of DAF-7 (IgG-DAF-noAmber). An amber codon was encoded in frame at the HC-DAF-7 junction to generate (IgG-DAF-Amber).

The pMOAV2 vector was based on the pSELECT-Jump-In (Thermo) vector containing CMV-pylRS expression cassette and 18 tandem repeats of the tRNApyl gene under control of the U6 snRNA promoter. The pCLD-puro-pylRS-tRNA vector was based on the pCLD vector containing puromycin resistance marker, CMV-pylRS expression cassette and 18 tandem repeats of the tRNApyl gene under control of the U6 snRNA promoter. The pRFP-GFPamb vector was the reporter construct encoding an RFP-GFP fusion containing an amber codon between the RFP and GFP fluorophores.

The methods for the generation of host cells capable of nnAA incorporation are known and have been described. In brief, CHO cells were transfected with pMOAV2 or pCLD-puro-pylRS-tRNA and subjected to a selection step for growth in hygromycin or puromycin (6.5 μg/ml) containing medium. Survivors were transfected with pRFP-GFPamb and grown in the presence of 2 mM nnAA for 16-24 hours. Isolates showing the best RFP:GFP ratios (C13-43) were selected for further analysis.

Alternately, to generate the I-21 host, a pool of GFP-RFP+ cells was sorted. Candidate hosts were further evaluated for their ability to incorporate nnAA in a target IgG and titers measured. The best candidates C13-43 and I-21 were identified.

To test amber suppression induced surface display of mAb, 1×10⁷ transfected cells expressing 1C1-DAF were centrifuged at 300 g for 5 minutes, resuspended in 10 ml fresh culture media containing lysine azide and incubated for 2 or 4 hours shaking at 120 rpm at 37° C. Following lysine azide treatment, 1×10⁶ cells were centrifuged at 300 g for 5 minutes, washed with FACS buffer (2% fetal bovine serum in 1×PBS) and stained with FITC-conjugated γ-chain antibody and APC-conjugated kappa light chain antibody (Life Technologies, Carlsbad, Calif.) for 15 min at room temperature. The surface stained cells were washed twice with FACS buffer and resuspended in FACS buffer for flow cytometry analysis in LSRII (BD Biosciences, San Jose, Calif.). Different concentrations of lysine azide were tested to determine the optimal concentration. Data analysis was performed using FlowJo software (Tree Star, Inc., Ashland, Oreg.).

Cells were seeded at 3×10⁵ cells/ml in 30 ml in-house culture media in 125 ml shake flask and grown at 37° C., 6% CO₂ and 120 rpm on an orbital shaking platform incubator. Cells were passaged twice a week following measurement of viable cell density and viability using ViCell automated cell counter (Beckman Coulter, Brea, Calif.).

Bulk and single cell sorting based on mAb surface display was performed using BD Influx cell sorter (BD Biosciences). Briefly, cells were treated with lysine azide for 2 or 4 hours following which 20×10⁶ and 1×10⁶ cells were harvested by centrifugation for bulk and single cell sorting respectively. The cells were washed and stained with FITC-conjugated γ-chain antibody using sorting buffer containing PBS, 0.5% recombinant human serum albumin (Sigma, St. Louis, Mo.), 5 mM EDTA (Life Technologies) and 25 mM HEPES (Calbiochem, San Diego, Calif.). For bulk sorting, stained cells were washed twice, resuspended in sorting buffer to a concentration of 1×10⁷ cells/ml and 2.5×10⁵ surface stained gated (based on high or low FITC fluorescence intensity) cells were deposited in 5 ml collection tubes containing culture media. The sorted cells were centrifuged, resuspended in 2.5 ml fresh culture media and plated in 6-well plates. For single cell sorting, the stained cells were washed, resuspended in sorting buffer to a concentration of 1×10⁶ cells/ml and one cell deposited per well of 384-well plates containing conditioned media.

The bulk and single sorted cells were expanded, propagated and finally seeded in fed-batch culture media for antibody production. The cultures received regular feeds for 12-14 days after which antibody titers in the cell culture supernatant were determined using Protein A biosensors in Octet QK384 (Pall ForteBio, Fremont, Calif.).

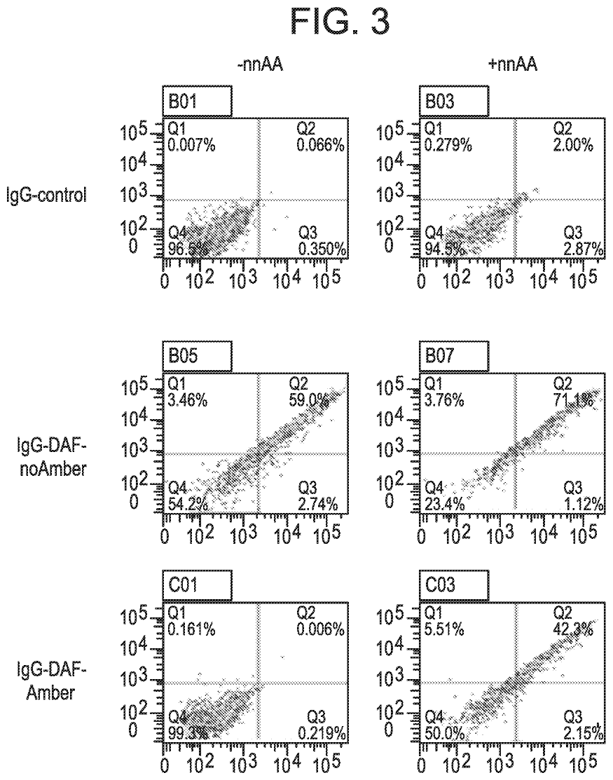

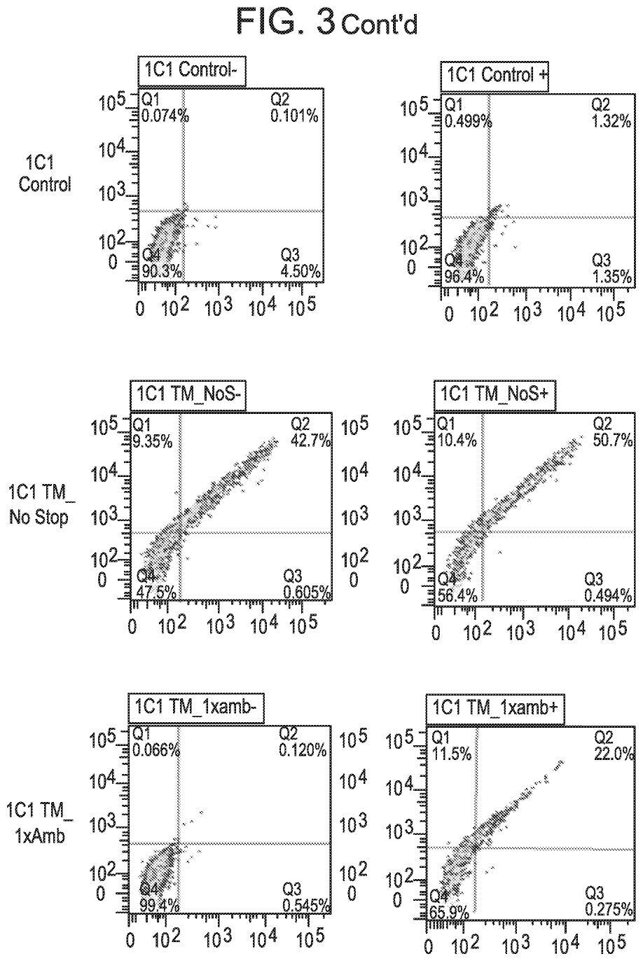

To establish whether a switchable IgG membrane anchor could be effectively displayed and visualized on the cell surface, the amber suppression host cells I-21 were transiently transfected with IgG expression plasmids and stable pools generated. Three different constructs were expressed in these cells encoding either a control IgG, an IgG containing a HC-Glycosylphosphatidylinositol membrane anchor (GPI-anchor) or an IgG containing the HC-GPI anchor that also contains an amber stop codon prior to and in-frame with the GPI cassette. To assess whether discrete membrane staining was observable transiently transfected I-21 cells were treated with lysine azide for 12 hours, immunostained for membrane bound LC and HC and analyzed by flow cytometry. The cells of interest were transfected with IgG-DAF-Amber, while cells transfected with IgG-control and IgG-DAF-noAmber were used as negative and positive control respectively. Flow cytometric analysis revealed no surface display for negative control cells (IgG-control), greater than 50% LC+HC+ population for positive control cells (IgG-DAF-noAmber) both in the presence and absence of lysine azide and 42% double positive cells with IgG-DAF-Amber in the presence of lysine azide. Importantly, in the absence of nnAA, surface staining of this construct was similar to the IgG control samples indicating no significant accumulation of IgG on the cell surface. These results show a clear and distinguishable accumulation of IgG-GPI at the cell surface (FIG. 3).

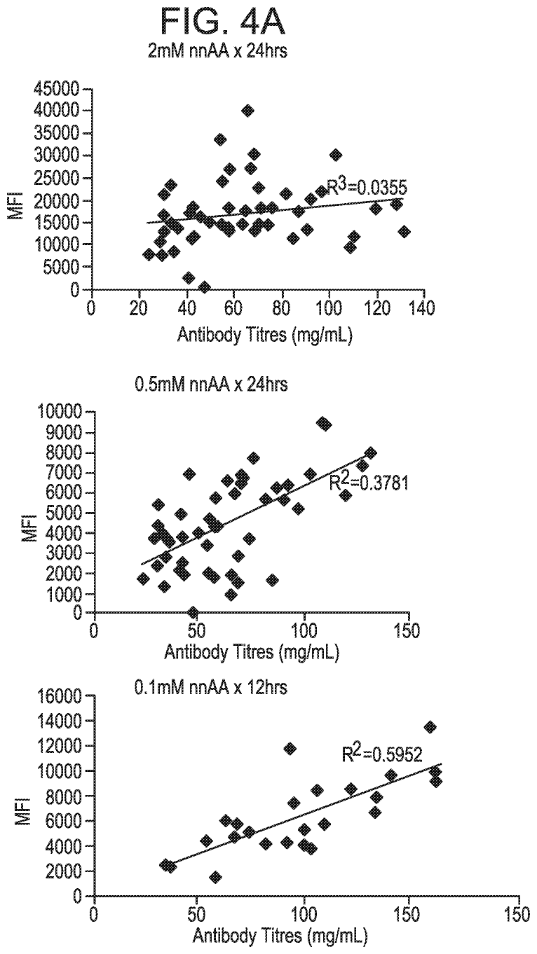

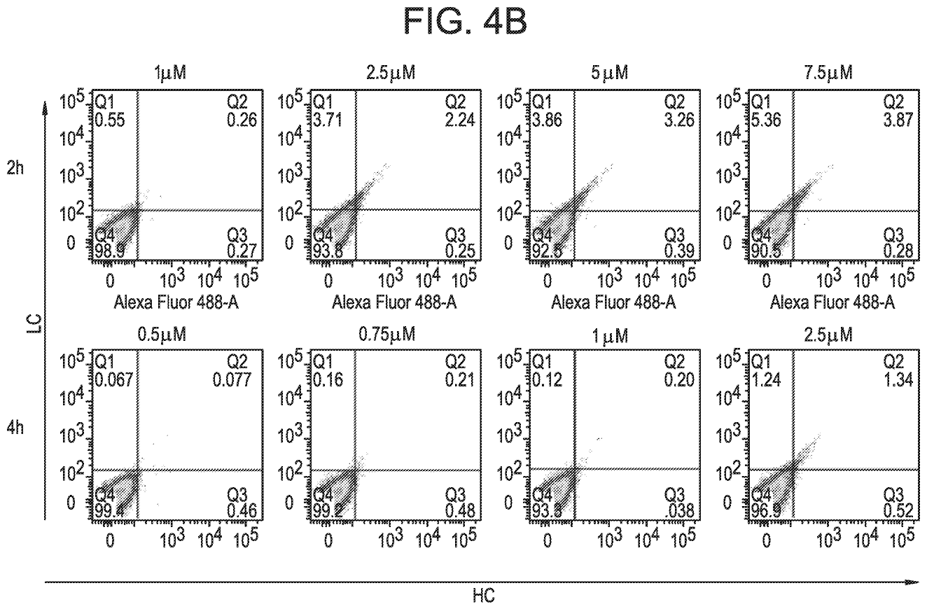

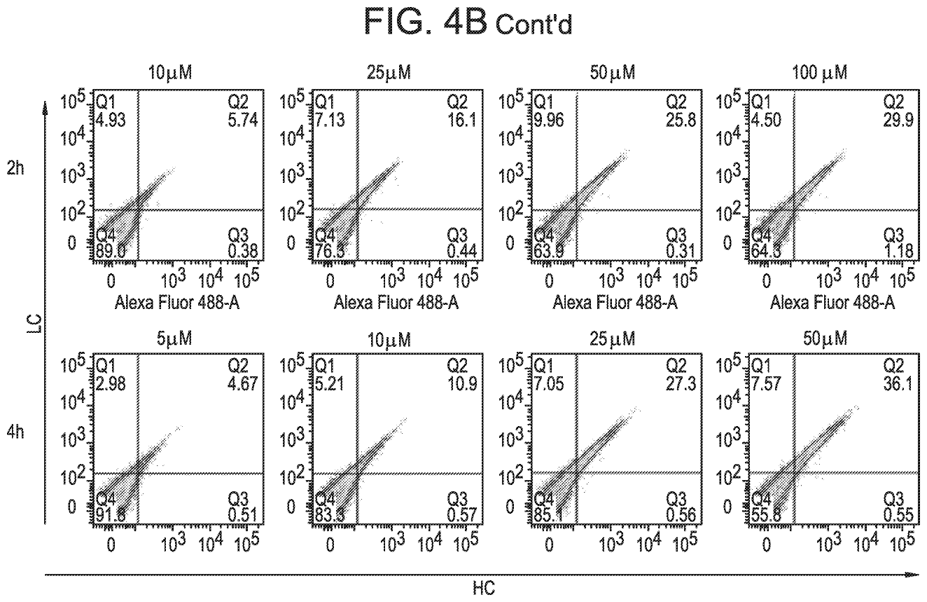

To determine whether this display was applicable to stable pools, surface binding was analyzed in stable pools of I-21 and C13-43 platform cells which had been transfected with the IgG-DAF-Amber construct. Following lysine azide treatment for 12 hours and immunostaining, marked surface display of IgG was observed in both I-21 and C13-43 stable pools as evident from the presence of LC+HC+ double positive population. A clear display of IgG-DAF fusions was observed in stable cells. Various concentrations of nnAA and times of treatment were examined to determine the effect of time and concentration of nnAA for optimal efficiency of amber suppression in the shortest time without saturation of the cell membrane. To determine the best conditions for surface display for the IgG-DAF-amber construct cells sorted from high surface display gates were collected from populations of cells activated by either 2 mM nnAA, 0.5 mM nnAA for 24 hours or 0.1 mM nnAA for 12 h. Our results show improved correlation of titre to MFI in cells activated under 0.1 mM nnAA for 12h indicating that an optimal activation of the system is needed to effectively discriminate low and high producers by this method (FIG. 4). Furthermore, it is likely that oprimization of these conditions will be necessary for each target to match their relative expression level. Further optimization with this target determined that 2-4 hours of 5-25 μM lysine azide treatment is sufficient to induce detectable cell surface binding of mAbs.

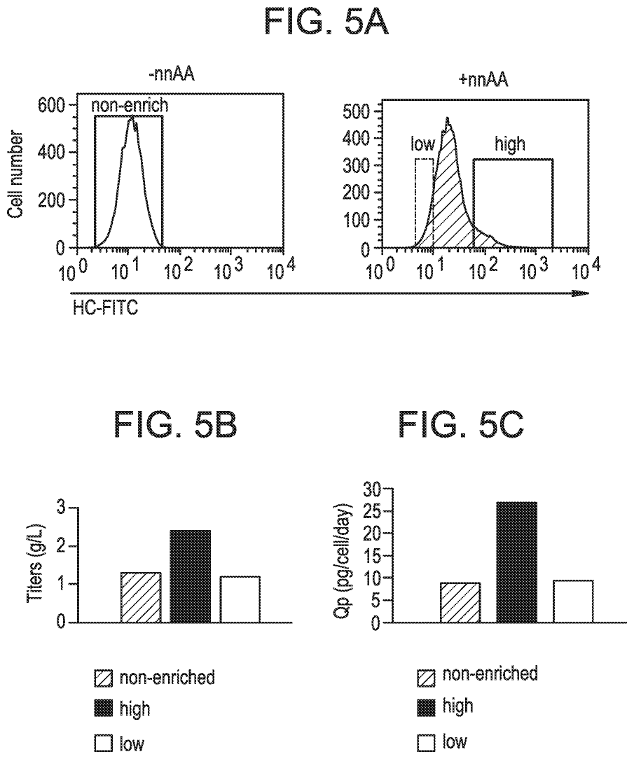

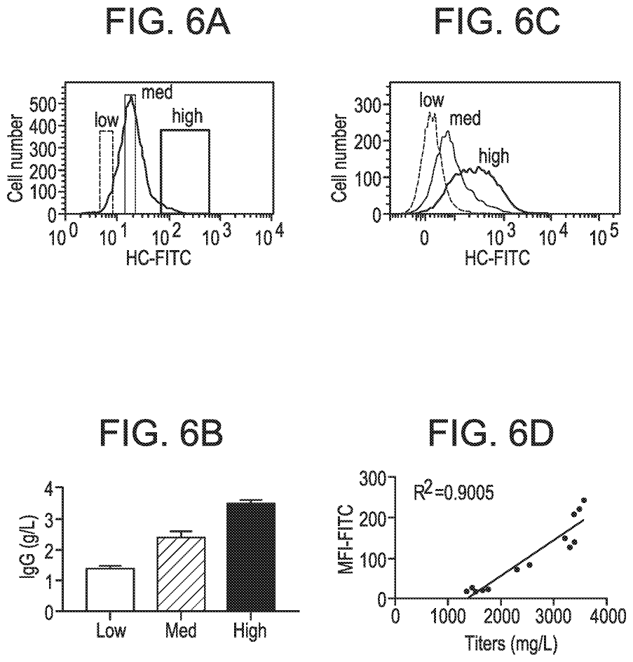

To assess whether the expression of membrane bound IgG correlated with secreted IgG titres, I-21 and C13-43 stable pools expressing IgG-DAF were treated with lysine azide, and sorted into two populations based on high and low expression levels of membrane-bound IgG detected by flow cytometry. The sorted cells were sub-cultured and their productivity was measured following 11 days of fed-batch culture in shake flasks. Cells selected for high levels of surface display showed improved expression levels over cells selected for low levels surface display or unselected populations. The I-21 pools had 2.4 and 1.2 g/L (FIG. 5) and the C13-43 pools had 3.4 and 1.5 g/L (FIG. 6) of titers from high and low surface display, respectively, suggesting that amber suppression dependent surface display is proportional to the secreted IgG in the medium. Moreover, FACS enrichment led to a 2-fold increase in productivity compared to non-enriched cells.

To further investigate the correlation between productivity and surface display, the C13-43 pools were sorted based on high, medium and low surface display and post sort cells were expanded for fed-batch culture as well as analysis for surface binding. When the titer values were plotted against the fluorescence intensity of membrane bound IgG, a significant correlation was observed with a correlation coefficient of 0.9005, FIG. 6, indicating that nnAA-induced surface display can be used as a representative of cell productivity.

Following lysine azide treatment of C13-43-IgG-DAF stable pools and surface staining, single cells were deposited into each well of 384 well plates based on low (bottom 5%), medium (mid 5%) and high (top 3%) fluorescence intensity of membrane bound IgG-HC. Untreated cells were cloned without any staining. A total of 283 clones (top 77, medium 50, bottom 84 and untreated 72) were screened in fed-batch cultures and analyzed for productivity. The clones obtained from top surface display gated region had significantly higher productivity compared to the bottom surface display clones. Moreover, the average productivity of the top clones was higher than the non-enriched clones.

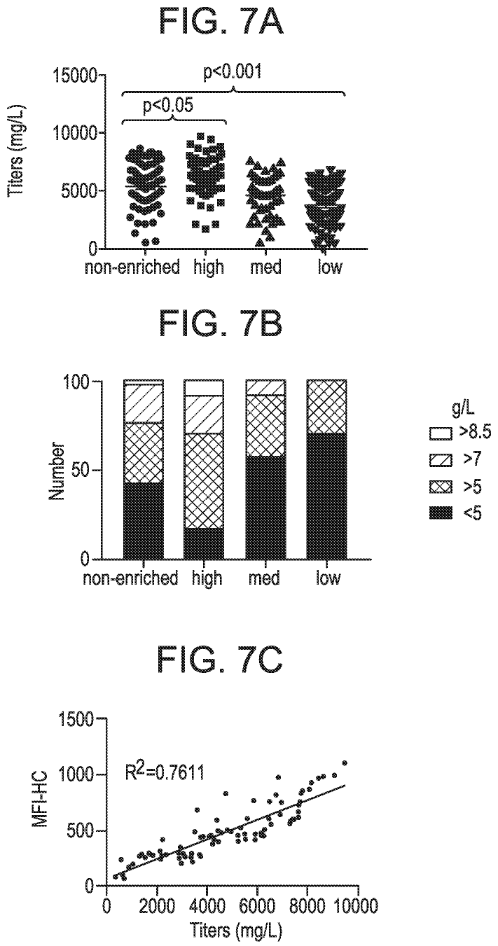

To determine the correlation between secreted and membrane-bound antibody of the clonal cells, 33 clones from each of high, medium and low surface display groups with various titer levels were selected for further analysis (FIG. 7). The individual clonal cells were treated with lysine azide, stained for surface binding of light and heavy chain and analyzed by flow cytometry. The titers of the clones when plotted against the median fluorescence intensity (MFI) of membrane-bound heavy chain antibody revealed a positive correlation (R²=0.7611) thereby strengthening the efficiency of this method and indicating that this strategy provide an effective tool for high throughput screening of the high producing cells in biomanufacturing process.

### Example 3—Difficult to Express Protein Expression

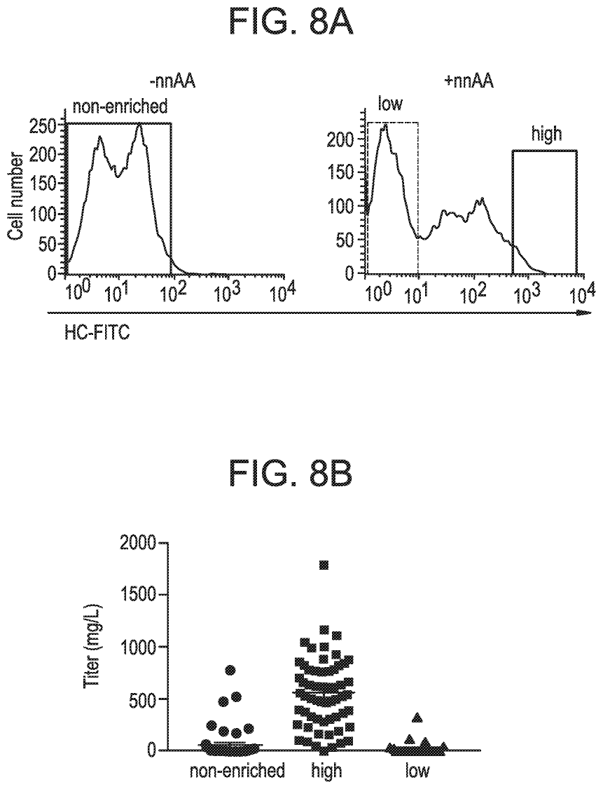

Complex recombinant molecules are emerging as the next generation of therapeutics. These highly engineered proteins, include bispecific antibodies (e.g. BiTES, DARTS, and IgG-scFvs) and fusion proteins that represent important new medicines with enhanced functionality for disease treatment. However, these molecules are often labeled as “difficult to express” because of low expression titer and low specific productivity. To address the bottleneck we investigated the use of the surface display method to isolate high producer cells for a difficult to express target. To do this, C13-34 cells were stably transfected with a plasmid encoding a proprietary bispecific antibody (MEDI-X) bearing the reversible GPI membrane anchor. MEDI-X, an IgG-scFv fusion protein, was selected in part because an extensive conventional screen had been recently conducted that resulted in the isolation of cell lines capable of 1 g/L yields. The transfected cells were subjected to surface display and selection from both high and low surface staining gates to determine whether this method could improve on the previously observed yields. Optimization of the surface display conditions identified 1 mM lysine azide and 4 hours of activation were sufficient to demonstrate surface display of this low expressing molecule. This condition was used for the sorting and selection of clones. A control population sorted without surface display was generated (non-enriched) in parallel. The recovered clones were expanded and their productivity was determined in 96 deep well plates by fed-batch culture (FIG. 8A). The top clone from the non-enriched population achieved a titre of ˜800 mg/L that is consistent with previous efforts. However, with surface display we saw, not only a significant improvement in the titres of the top expressor (up to 1.8 g/L), but also eighteen additional clones with titres above 800 mg/L, including five clones with yields exceeding 1 g/L (FIG. 8B). These data illustrate the utility of this approach for the enrichment and selection of high expressor clones (FIG. 8).

What is claimed is:

1. A method of screening a population of transgenic cells for cells that produce a protein of interest, the method comprising,
a) culturing the transgenic cells in culture conditions that permit protein synthesis, wherein the cell culture conditions comprise at least one non-natural amino acid (nnAA) in the cell culture medium, and
b) determining which of the transgenic cells produce the protein of interest,
wherein the transgenic cells comprises (i) a polynucleotide encoding a fusion protein comprising at least one nnAA, a first domain coding a protein of interest, and a second domain coding a domain that facilitates detection of the transgenic cell that produces the protein of interest when the transgenic cell expresses the second domain, and (ii) at least one orthogonal tRNA and at least one orthogonal tRNA synthetase that accepts the at least one nnAA and recognizes the at least one orthogonal tRNA.

2. The method of claim 1, wherein the polynucleotide encoding the at least one nnAA is an amber stop codon that supports suppression of translation termination in the presence of the nnAA.

3. The method of claim 1, wherein the at least one nnAA is pyrrolysine, the orthogonal tRNA is pyrrolysyl-tRNA (tRNA-Pyl), and the orthogonal tRNA synthetase is pyrrolyl-tRNA synthetase (PyrlRS).

4. The method of claims 1-3, wherein the second domain of the fusion protein comprises a transmembrane protein domain, a cell membrane anchor domain, or a tag.

5. The method of any of claims 1-4, wherein the transgenic cells are mammalian cells.

6. The method of claim 5, wherein the transgenic cells are CHO cells, HEK293 cells, PERC6 cells, COS-1 cells, HeLa cells, VERO cells or mouse hybridoma cells.

7. The method of any of claims 1-4, wherein the transgenic cells are prokaryotic cells.

8. The method of any of claims 1-7, wherein the fusion protein comprises a linker peptide sequence between the first and second domains.

9. The method of any of claims 1-8, wherein the second domain is a transmembrane protein domain.

10. The method of any of claims 1-8, wherein the second domain is a cell membrane anchor domain is a glycosylphosphatidylinositol (GPI) signal peptide that promotes anchoring the protein of interest to a GPI moiety present in the cell membrane.

11. The method of claim 10, wherein the GPI signal peptide is a GPI signal peptide from the decay accelerating factor 7 protein (DAF-7).

12. The method of any of claims 1-11, wherein the first domain of the fusion protein is an antibody or antibody fragment.

13. The method of any of claims 1-12, wherein determining if the protein of interest is produced in or on the transgenic cells comprises determining levels of the protein of interest that are displayed on the surface of the transgenic cells to determine which of the transgenic cells produces the protein of interest at higher levels compared to other transgenic cells in the population of transgenic cells.

14. The method of claim 13, wherein the transgenic cells that produce higher amounts of the protein of interest are separated from the transgenic cells that produce lesser amounts of the protein of interest.

15. The method of any of claim 14, wherein separating the transgenic cells comprises using fluorescent activated cell sorting (FACS).

16. The method of claim 14 or 15, wherein the separated higher producing transgenic cells are subsequently cultured in cell culture conditions that permit protein synthesis.

17. The method of claim 16, wherein the higher producing transgenic cells are cultured in a cell culture environment in the absence of the at least one nnAA, wherein culturing the transgenic cells in the absence of the at least one nnAA results in the production of the protein of interest without the second domain of the fusion protein.

18. The method of claim 17, further comprising isolating the protein of interest from the cell culture environment.

19. The method of any of claims 1-8, wherein the second domain is protein tag.

20. The method of claim 19, wherein the tag is an affinity, epitope, or fluorescent tag.

21. The method of any of claim 1-8, 19, or 20, wherein the first domain of the fusion protein is a complex membrane protein (CMP).

22. The method of claim 21, wherein determining if the protein of interest is produced in or on the transgenic cells comprises detecting a fluorescent signal emitted from a fluorescent protein to determine which transgenic cells are producing the protein of interest.

23. The method of claim 22, wherein the transgenic cells that produce the protein of interest are separated from the transgenic cells that do not produce the protein of interest.

24. The method of any of claims 1-23, wherein separating the transgenic cells that do not produce the protein of interest comprises fluorescent activated cell sorting (FACS).

25. The method of claim 23 or 24, wherein the separated transgenic cells that produce the protein of interest are subsequently cultured in cell culture conditions that permit protein synthesis.

26. The method of claim 25, wherein the transgenic cells that produce the protein of interest are cultured in a cell culture environment in the absence of the at least one nnAA, wherein culturing the transgenic cells in the absence of the at least one nnAA results in the production of the protein of interest without the second domain of the fusion protein.


SEQUENCE LISTING (4 sequences)

SEQ ID NO: 1 - protein, 15 aa, Artificial Sequence
  Synthetic peptide
GLNDIFEAQKIEWHE

SEQ ID NO: 2 - protein, 9 aa, Artificial Sequence
  Synthetic peptide
TETSQVAPA

SEQ ID NO: 3 - protein, 14 aa, Artificial Sequence
  Synthetic peptide
GGSGGSGGSGGSGG

SEQ ID NO: 4 - protein, 7 aa, Artificial Sequence
  Synthetic peptide
DYDDDDK